(19) United States
(12) Patent Application Publication
(10) Pub. No.: US 2008/0103057 A1
(43) Pub. Date: May 1, 2008

(54) Method for multi-color fab labeling of antibodies in a complex sample

(76) Inventors: Paul J. Utz, Portola Valley, CA (US); Michael G. Kattah, Stanford, CA (US); Golnaz Alemi, Palo Alto, CA (US); Donna L. Thibault, Redwood City, CA (US); Imelda M. Balboni, Mountain View, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/895,049

(22) Filed: Aug. 22, 2007

Related U.S. Application Data
(60) Provisional application No. 60/839,838, filed on Aug. 23, 2006.

(51) Int. Cl. C40B 30/04; G01N 33/563

(52) U.S. Cl. 506/9; 436/512

(57) ABSTRACT

Monovalent Fab labeling reagents are conjugated to spectrally-resolvable fluorescent dyes. Each of these reagents are incubated with a patient sample comprising antibodies, to allow the monovalent Fab fragments to bind and label the antibodies. The patient samples are then used directly to contact and stain antigen, e.g. an antigen microarray, histologic section, tissue microarrays, cells, etc.

### CROSS-REFERENCE TO RELATED APPLICATIONS

This applications claims benefit of priority to U.S. provisional application 60/839,838, filed Aug. 23, 2006, which is herein incorporated by reference.

### BACKGROUND OF THE INVENTION

Achieving reproducible, specific staining is widely sought in immunological determinations for measuring the presence of specific antibodies in complex biological samples such as serum or cerebrospinal fluid. Often the specific binding of a detection reagent to its target antigen cannot be distinguished from non-specific binding to other structures that are unrelated to the antigen of interest; and signal strength can be variable, making reproducibility difficult. Ideally, detection reagents used in immunological determinations provide for specific and reproducible signals.

Antibodies that are reactive against specific self-antigens are characteristic of many autoimmune diseases. These antigens include a diverse group of cell-surface, cytoplasmic, and nuclear antigens. The detection of autoantibody specificity is of particular clinical interest for many diagnostic assays. For example, disease profiling has been accomplished with planar protein microarrays by binding autoantibodies to a large panel of potential autoantigens in a variety of autoimmune diseases. Antigen microarrays have also been used to guide antigen-specific tolerizing therapy in models of disease, to identify clinical subtypes of rheumatoid arthritis with respect to autoreactivities and disease severity, and for identification of autoreactivities in sera from lupus patients that correlate positively or negatively with disease severity. Variations of this technology have also been used profile the antibody repertoire in patients with prostate cancer and in patients suffering from allergies. However, while assays utilizing multiplexed samples provide useful information in antibody profiling studies, improvements in reproducibility and sample normalization are required before they become a common clinical tool.

Current methods for detecting antibodies bound to antigen microarrays include single and double color analysis. The single-color method involves probing an array with unlabeled serum followed by detection with a secondary antibody conjugated to a fluorophore. This approach provides simplicity and standardization with respect to fluorophore, but it suffers from variability between array features, arrays, samples, and laboratories. A two-color approach is an attractive alternative that can control for some of these sources of variability. Reports have described methods of two-color protein microarrays, but these techniques suffer from inherent limitations of N-hydroxysuccinimidyl (NHS)-ester chemical coupling procedures that were used. The drawbacks of this strategy include expense, labor, highly variable modification efficiency due to hydrolytic side reactions, and potentially reduced binding due to modification of primary amines.

A desirable approach to improve the reproducibility and standardization of patient antibody sample assays could provide a simple and inexpensive method of multicolor antibody labeling. The present invention addresses this need.

### SUMMARY OF THE INVENTION

A multi-color Fab labeling method is provided, which allows multiple samples to be applied simultaneously to the same substrate. This labeling method improves reproducibility and reliably detects changes in antibody levels. Monovalent Fab labeling reagents are conjugated to spectrally-resolvable fluorescent dyes. Each of these reagents is incubated with a patient sample comprising antibodies, to allow the monovalent Fab fragments to bind and label the antibodies. The patient samples are then used directly to contact and stain antigen, e.g. an antigen microarray, histologic section, tissue microarrays, cells, etc.

In certain embodiments of the invention, each Fab labeling reagent conjugated to spectrally-resolvable fluorescent dye is contacted with a separate patient sample, where the patient samples may be the same or different. Where the patient samples are the same, each sample may be a duplicate, or aliquot, from a patient specimen. Where the samples are different, the samples may be obtained from a single patient as different time points, e.g. during therapy, prior to diagnosis, etc., or may be from a test patient and a control sample, e.g., from a patient known to be negative or positive from disease.

An advantage of the labeling methods of the invention is that it provides for reproducible labeling of small amounts of patient samples. The primary antibodies are not chemically modified and should therefore better retain antigen-binding ability. Finally, these inexpensive labeling reagents can be generated in large amounts to improve consistency.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent application publication with color drawing(s) will be provided by the U.S. Patent and Trademark Office upon request and payment of the necessary fee.

FIG. 1. Two-color Fab labeling for probing one array with different serum samples. (a) Single-color method schematic of probing two arrays with two different samples using a fluorescent secondary antibody. (b) Two-color Fab method schematic of probing one array with two samples. We pre-incubate samples with fluorescent secondary monovalent Fab fragments and mix before probing. (c) and (d) We spiked mouse monoclonal anti-MPO (2 μg) or monoclonal anti-PR3 (2 μg) into normal mouse serum (2 ul containing 20 ug total IgG). Samples were labeled with 30 μg of either Cy3 or Cy5-labeled GAM Fab fragments for a molar ratio of 4.5:1 Fab:IgG. (c) Scanned image of the array probed with anti-MPO serum (Cy3) and anti-PR3 serum (Cy5). (d) Scanned image of the array probed with anti-MPO serum (Cy5) and anti-PR3 serum (Cy3). Antigens: (1) MPO-2, (2) MPO-1, (11) MPO-3, (3) PR3-2, (4) PR3-1, (23) PR3-3, (24) anti-IgG. (e) and (f) We spiked human anti-Ro/SSA (1 μg) and anti-La/SSB (1 μg) into normal human serum (2 ul containing 40 ug of total IgG). Samples were labeled with 40 ug Alexa555 or Alexa647-GAH Fab fragments for a molar ratio of 3:1 Fab:IgG. (e) Scanned image of the array with anti-Ro/SSA serum (Alexa647) and anti-La/SSB serum (Alexa555). (f) Scanned image of the array with anti-Ro/SSA serum (Alexa555) and anti-La/SSB serum (Alexa647). Antigens: (10) La/SSB, (13) Ro/SSA, (24) anti-IgG, (18) U1A. Emission at 532 nm (Cy3 or Alexa555) is pseudocolored blue, emission at 635 nm (Cy5 or Alexa647) is pseudocolored yellow, and emission of equal intensity in both channels is pseudocolored white.

FIG. 2. Signal Intensity, Sensitivity, and Dynamic Range. (a) and (b) We spiked monoclonal anti-La/SSB into normal mouse serum (20 μg IgG) at five concentrations 10% (2 μg), 1% (200 ng), 0.1% (20 ng), 0.01% (2 ng), and 0% (0 ng). Alexa dyes with an MSR of 1.5 dye molecules/Fab fragment were used at a molar ratio of 6:1 Fab:IgG. The two-color data are from a self-self array; the single color data from one array. (a) MFI-B of the La/SSB features on the autoantigen arrays plotted against anti-La concentration. Error bars represent 95% confidence intervals, n=12. (b) Intraslide % CV of the La/SSB features on the two-color autoantigen arrays plotted against anti-La concentration. (c) and (d) We spiked monoclonal antibodies directed against PR3 and MPO into normal mouse serum at serial three-fold dilutions in opposing gradients such that the highest anti-MPO reactive sample had the lowest anti-PR3 reactivity and vice versa. We calculated the log₃ change relative to a middle value (anti-MPO and anti-PR3 at approximately 0.3% of total serum IgG) to monitor up- and down-regulation of autoreactivity. Error bars represent 95% confidence intervals, n=3.

FIG. 3. Autoantibody profiling of mouse serum and Ribosomal P autoreactivity in the pristane model of lupus. (a) Heat map representations of log₂ of 635/532 nm ratios for each antigen. We tested mouse serum from the pristane group pre-treatment (Pristane-pre), pristane group 20 weeks after treatment (Pristane-post), PBS group pre-treatment (PBS-pre), and PBS group 20 weeks after treatment (PBS-post). (1) Pristane-post (Alexa647-Fab) with pristane-pre (Alexa555-Fab); (2) PBS-post (Alexa647-Fab) with PBS-pre (Alexa555-Fab). Positive log₂ values (ratios>1) are pseudocolored yellow and negative log₂ values (ratios<1) are pseudocolored blue. (b) and (c) Autoantibodies to whole recombinant P0 as obtained by ELISA using serum from pristane-treated and PBS-treated mice. (b) Scatter plot of PBS and pristane-treated mice. Bars show the mean optical density values for each group and the broken line represents mean of PBS treated animals plus 3 standard deviations. (c) Autoantibodies to whole recombinant P0 as obtained by Ribo-P ELISA. Error bars represent 95% confidence intervals, n=3. (d) Immunoblot of recombinant P0 fractionated by SDS-PAGE and probed with serum using slot blot device. Lanes are human anti-Ribo-P reactive serum (Anti-Ribo P), and serum from mice in the following groups: PBS-pre, PBS-post, Pristane-pre, and Pristane-post. Positions of molecular weight (m.w.) markers in kilodaltons (kD) are indicated on the left. Full-length blot presented online (Supplementary FIG. 4b). Order of samples on ELISA graph (c) and immunoblot (d) is the same.

FIG. 4. Fold-changes from murine and human dye-swap experiments. (a) Average of the log 2 of the (635/532 nm) and (532/635 nm) ratios from the murine dye-swap experiments in FIGS. 1c, d. (b) Average of the log 2 of the ratios from the human dye-swap experiments in FIGS. 1e, f. Array features (1)-(24) correspond to bar graph columns (1)-(24) for the murine (a) and human (b) experiments, respectively. Error bars represent 95% confidence intervals, n=12, (*) signifies changes greater than two-fold.

FIG. 5. Cross-labeling of Fab fragments. (a) MFI-B in 635 nm and 532 nm channels for PR3 antigen features from two arrays probed at room temperature and one array probed at 4° C. using the two-color Fab method. Array 1—normal mouse serum (NMS) (Alexa647) and NMS (Alexa555); Array 2—2 ug anti-PR3 in NMS (anti-PR3) (Alexa647) and NMS (Alexa555) Array 3-anti-PR3 (Cy5) and 2 ug anti-MPO in NMS (anti-MPO) (Cy3). 20 ug IgG in 2 ul of NMS for each sample. (b) Relative 1093 of (635/532) ratio and (c) MFI-B in 635 nm and 532 nm channels at different time-points comparing serum with 3 times higher anti-PR3 reactivity and 3 times lower anti-MPO reactivity (Alexa647) than a reference sample (Alexa555). Error bars represent s.e.m., n=3.

FIG. 6. Correlation of single-color and two-color Fab methods with conventional ELISA. We spiked monoclonal antibodies directed against PR3 and MPO into normal mouse serum at serial three-fold dilutions and assayed by single-color arrays (MFI-B, right axis), two-color arrays (635/532 nm Ratio, left axis), and conventional ELISA (OD₄₅₀, x-axis). Data are representative of two experiments.

FIG. 7. Anti-ribosomal P reactivity by western-blot and immunoprecipitation in pristane-treated BALB/c mice. (a) Immunoprecipitation using sera from pristane-treated and PBS-treated BALB/c mice. Lanes are 9A9 anti-U1A/U2B″ monoclonal antibody (anti-U1A/U2B″), human anti-Ribo-P serum (anti-Ribo P), pristane-pre, pristane-post, PBS-pre, and PBS-post. Radiolabeled EL4 cell extract was immunoprecipitated and developed by autoradiography (bottom) or probed using anti-Ribo P serum (top). Positions of m.w. markers in kD are indicated on the right. (b) Full-length immunoblot of FIG. 3d.

### DETAILED DESCRIPTION OF THE EMBODIMENTS

Monovalent secondary Fab fragments are conjugated to spectrally-resolvable fluorescent dyes. Each of these reagents is incubated with a patient sample comprising antibodies, to allow the monovalent Fab fragments to bind and indirectly label the antibodies. The patient samples are then used directly to contact and stain antigen, e.g. an antigen microarray, histologic section, cells, etc. The amount of Fab reagent, dilution of sample, and incubation time for staining the sample are flexible and can be easily adjusted according to the specific application. The results of such assays find a variety of uses, e.g. selection of therapy for autoimmune disease, determination of exposure to pathogens, classification of allergic responses, and the like, as known in the art.

Multi-color labeling of antibody-containing samples can be utilized in binding to the same or to separate arrays, in order to assay the level of binding in a patient sample compared to a reference sample, or as internal controls for the level of binding of a single sample. From the ratio of one color to the other, for any particular array element, the relative abundance of antibodies with a particular specificity in the two samples can be determined. In addition, comparison of the binding of the two samples provides an internal control for the assay. Competitive assays are well known in the art, where a competing antibody of known specificity, or an epitope containing molecule, may be included in the binding reaction. The competitive nature of the binding in the two-color method, in contrast to single-color methods, makes the multi-color approach more resistant to variation between arrays and array elements.

Fab. The term Fab, as used herein, refers to a monovalent antibody fragment comprising antibody variable region domains, usually the complexed heavy and light chain variable domains and one constant kappa or lambda domain. As is known in the art, the enzyme papain cleaves antibodies into two Fab fragments and an Fc fragment. The two Fab fragments produced each recognize the antigen specifically with their variable region. The papain cleavage site is above the hinge region containing the disulfide bonds that join the heavy chains, but below the site of the disulfide bond between the light chain and heavy chain. The fragments thus produced can be purified by gel filtration, ion exchange, or affinity chromatography. Protocols for antibody digestion and purification of antibody fragments can be found in Antibodies: A Laboratory Manual, E. Harlow and D. Lane, ed., Cold Spring Harbor Laboratory, Cold Spring Harbor, N.Y., 1988. Each Fab fragment is monovalent whereas the original molecule was divalent or multivalent. Since the Fab fragments are monovalent, there is no risk of cross-linking different antibodies during the labeling step.

The Fab fragments generated by papain cleavage correspond to the two identical arms of the antibody molecule, containing the complete light chains paired with the V_{H} and C_{H}1 domains of the heavy chains. The Fab fragments may be proteolytically generated, or alternatively, genetic engineering techniques may be used to generate truncated antibody polypeptides, e.g. comprising only the heavy and light chain variable regions; the variable domain of a heavy chain linked by a stretch of synthetic peptide to a variable domain of a light chain; and the like.

Fab polypeptides used in the methods of the invention specifically bind to the antibodies present in the patient sample, e.g. they may be anti-human antibodies; anti-mouse antibodies; anti-rat antibodies; anti-human Fc; and the like. Such labeling reagents are known and used in the art, and are commercially available. Readily available sources include goat, mouse, rat, rabbit, etc., anti-species antibodies, which may be available as Fab fragments, or may be readily cleaved to provide for monovalent binding fragments.

In addition to labeling reagents that are broadly reactive with antibodies in a patient sample, the patient sample may be selectively labeled, e.g. using a Fab reagent that is isotype or subclass specific, e.g. anti-IgG, anti-IgM, anti-IgE, anti-IgA, anti-IgG1, anti-IgG2a, anti-IgG2b, anti-IgG3, anti-IgG4, and the like. Labeling with such reagents permits further refinement of the analysis, e.g. in the determination of antibodies present in a sample that react with an antigen of interest, vs. antibodies of a specific isotype or subclass that react with an antigen.

In an alternative embodiment, the monovalent detection reagent is other than an antibody, and comprises a labeled, monovalent entity that binds IgG at a high affinity, e.g. protein A, protein G, an evolved recombinant protein, an evolved DNA aptamer, etc.

Spectrally resolvable label. Useful labels include fluorochromes, e.g. Cy2, Cy3, Cy5, fluorescein isothiocyanate (FITC), rhodamine, Texas Red, phycoerythrin, allophycocyanin, 6-carboxyfluorescein (6-FAM), 2′,7′-dimethoxy-4′,5′-dich-loro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)). Useful labels also include radioisotopes, bar codes, quantum dots, and the like. A reporter used to label one of the Fab reagents will be selected so as to emit a signal at an excitation and/or emission wavelength detectably distinct from that of the reporter used to label the other Fab reagent(s), i.e. the two labels are spectrally resolvable. Methods of conjugating such labels to protein reagents are known in the art.

Patient samples. Biological samples from which patient samples comprising antibodies may be collected include blood and derivatives therefrom, e.g. serum, plasma, fractions of plasma, etc. Other sources of samples are body fluids such as synovial fluid, lymph, cerebrospinal fluid, bronchial aspirates, and may further include saliva, milk, urine, and the like.

Antibody containing samples may include the presence of autoantibodies, which are any antibody that recognizes or binds a self-antigen or self-epitope. Self-antigens or self-epitopes include polypeptides, proteins, peptides, lipids, polysaccharides, and modifications of these self-antigens that are encoded within the genome or produced within an organism.

Mammalian species that provide samples for analysis include canines; felines; equines; bovines; ovines; etc. and primates, particularly humans. Animal models, particularly small mammals, e.g. murine, lagomorpha, etc. may be used for experimental investigations. Animal models of interest include those for models of autoimmunity, graft rejection, and the like.

Antigen. The Fab labeled patient samples are used to contact and stain antigen, e.g. an antigen microarray, histologic section, cells, etc. In some embodiments, the antigen is provided as an array of antigens or epitopes. Antigens of interest include, without limitation, self antigens, allergens, infectious agents, bioterrorism agents, and the like.

An array is a collection of addressable elements. Such elements can be spatially addressable, such as arrays contained within microtiter plates or printed on planar surfaces where each element is present at distinct X and Y coordinates, planar samples of tissue sections comprising antigen, etc. Alternatively, elements can be addressable based on tags, beads, nanoparticles, or physical properties. Microarrays can be prepared according to the methods known to the ordinarily skilled artisan (See for example, U.S. Pat. No. 5,807,522; Robinson et al. (2002) Nature Medicine 8:295-301; Robinson et al. (2002) 46:885-93). Arrays as used herein refers to any biologic assay with multiple addressable elements. In one embodiment the addressable elements are antigens. As used herein, elements refer to any antigen that can be bound by an antibody.

The portion of the antigen bound by the antibody is referred to as an epitope. As used herein, an epitope is the portion of the antigen that is sufficient for high affinity binding. Where the antigen is a protein, generally a linear epitope will be at least about 7 amino acids in length, and not more than about 15 to 22 amino acids in length. However, antibodies may also recognize conformational determinants formed by non-contiguous residues on an antigen, and an epitope can therefore require a larger fragment of the antigen to be present for binding, e.g. a protein or ribonucleoprotein complex domain, a protein domain, or substantially all of a protein sequence. In other instances, e.g. haptens, the epitope can be a very small molecule, e.g. digoxin; digoxigenin, etc.

In one embodiment, an array is synthesized or spotted onto a planar substrate, producing, for example, microarrays, where a large number of different molecules are densely laid out in a small area, e.g. comprising at least about 400 different elements per cm², and may be 1000 elements per cm², or as many as 5000 elements per cm², or more. Less dense arrays, such as may be found in ELISA or RIA plates where wells in a plate each contain a distinct antigen, may comprise from about 96 elements per plate, up to about 100 elements per cm², up to the density of a microarray. Other spatial arrays utilize fiber optics, where distinct antigens are bound to fibers, which can then be formed into a bundle for binding and analysis. Methods for the manufacture and use of spatial arrays of polypeptides are known in the art. Articles include Joos et al. (2000) Electrophoresis 21(13):2641-50 describing a microarray-based immunoassay containing serial dilutions of antigens; Roda et al. (2000) Biotechniques 28(3):492-6 describing a system obtained by adapting a commercial ink-jet printer and used to produce mono- and bidimensional arrays of spots containing protein on cellulose paper; and Ge (2000) Nucleic Acids Res 28(2):e3 describing a universal protein array system for quantitative detection of protein-protein, protein-DNA, protein-RNA and protein-ligand interactions. See also, Mendoza et al. (1999) “High-throughput microarray-based enzyme-linked immunosorbent assay (ELISA)” Biotechniques 27:778-780; and Lueking et al. (1999) “Protein microarrays for gene expression and antibody screening” Anal. Biochem. 270:103-111.

One embodiment of an array is an antigen array. An antigen array as used herein, refers to a spatially separated set of discrete molecular entities capable of binding to antibodies which are arranged in a manner that allows identification of the specificity of the antibodies contained within the patient sample. In other words, a set of target antigens having distinct sequences, three dimensional shapes, or molecular structures, where each target antigen is coded for identification. The array may comprise one or more of proteins, polypeptides, peptides, RNA, DNA, lipid, glycosylated molecules, polypeptides with phosphorylation modifications, and polypeptides with citrulline modifications, aptamers, other molecules, and other molecules, where different classes of molecules may be combined in an array.

### Methods of Analysis

A multi-color Fab labeling method is provided, which allows multiple samples to be applied simultaneously to the same substrate. The methods utilize as a labeling reagent monovalent Fab fragments that are specific for antibodies present in a patient sample, typically being specific for constant regions of the patient sample antibodies, e.g. anti-human antibodies; anti-human IgG, etc. The labeling reagent may be prepared by recombinant methods, or isolated from animal serum and cleaved to provide for monovalent binding fragments.

A first Fab labeling reagent is labeled with a first label, e.g. a fluorochrome; and a second Fab labeling reagent is labeled with a second label, e.g. fluorochrome, where the first and the second labels are spectrally resolvable. Optionally, the labeling reagents are purified prior to use to eliminate excess fluorochrome, residual multivalent binding fragments, and the like. The steps may be repeated for additional labeling reagents, where each label will be spectrally resolvable from the others.

A first Fab labeling reagent comprising a label, e.g. fluorochrome is contacted with a first patient sample, and a second Fab labeling reagent comprising a spectrally resolvable label, e.g. fluorochrome is contacted with a second patient sample, where the two patient samples may be the same or different. Additional samples are optionally labeled with third, fourth, etc. reagents. Where the patient samples are the same, each sample may be a duplicate, or aliquot, from a patient specimen. Where the samples are different, the samples may be obtained from a single patient as different time points, e.g. during therapy, prior to diagnosis, etc., or may be from a test patient and a control sample, e.g. from a patient known to be negative or positive from disease.

The labeling reagents are incubated with the patient sample for a period of time and at a concentration sufficient to label substantially all or most of the antibodies present in the patient sample, where incubation may be at a temperature of from about 40 to about 37°, for a period of time ranging from around about 5 minutes to about 30 minutes. The labeled patient samples are used to contact and stain antigen, e.g. an antigen microarray, histologic section, cells, etc.

The patient samples may be obtained for a variety of purposes, including disease diagnosis; time courses that follow the progression of disease; comparisons of different patients at similar disease stages, e.g. early onset, acute stages, recovery stages, etc.; tracking a patient during the course of response to therapy, including drug therapy, vaccination and the like. Data from animal patients, e.g. mouse, rat, rabbit, monkey, etc. may be compiled and analyzed in order to provide databases detailing the course of disease, antigens involved in diseases, etc.

In a typical assay, a patient sample containing antibodies is physically contacted with antigen, e.g. antigen present in an array format, under conditions that permit high affinity binding, but that minimize non-specific interactions. In one embodiment, patient samples are pippeted onto the array or into a space containing the addressable elements. The array is washed free of unbound material, and the presence of bound antibodies is detected, and correlated with the cognate antigen.

Generally assays will include various reference samples, e.g. negative and positive controls, as known in the art. These may include positive controls of “spiked” samples with known autoantibodies, patients with known disease, and the like. Negative controls include samples from normal patients, animal serum, and the like. Binding of the antibody containing sample to an antigen array is accomplished according to methods well known in the art. The binding conditions and washes are preferably carried out under conditions that allow only high affinity binding partners to be retained.

Arrays can be scanned to detect binding of antibodies, e.g. using a scanning laser microscope as described in Shalon et al., Genome Res. 6:639 (1996). A separate scan, using the appropriate excitation line, is performed for each of the fluorophores used. The digital images generated from the scan are then combined for subsequent analysis. For any particular array element, the ratio of the signal from one sample is compared to the fluorescent signal from the other sample, and the relative abundance determined.

The antigen or epitope readout may be a mean, average, median or the variance or other statistically or mathematically-derived value associated with the measurement. The antigen or epitope readout information may be further refined by direct comparison with the corresponding reference or control pattern. A binding pattern may be evaluated on a number of points: to determine if there is a statistically significant change at any point in the data matrix; whether the change is an increase or decrease in the epitope binding; whether the change is specific for one or more physiological states, and the like. The absolute values obtained for each epitope under identical conditions will display a variability that is inherent in live biological systems and also reflects individual antibody variability as well as the variability inherent between individuals.

In some embodiments, kits are provided for the methods of the invention, where such kits will usually include a first and a second Fab labeling reagent, where the first comprises a fluorochrome that is spectrally resolved from a fluorochrome present on the second. The kit may further comprise arrays for use in staining, software for analysis, control antibody reagents, and the like.

The following non-limiting examples are illustrative of the present invention. While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### EXPERIMENTAL

Antigen microarrays hold great promise for profiling the humoral immune response in the setting of autoimmunity, allergy, and cancer. This approach involves immobilizing antigens on a slide surface and then exposing the array to biological fluids containing immunoglobulins. Although these arrays have proven extremely useful as research tools, they suffer from multiple sources of variability. In order to address these issues, we have developed a novel two-color Fab labeling method that allows two samples to be applied simultaneously to the same array. This straightforward labeling approach improves reproducibility and reliably detects changes in autoantibody levels. Using this technique we profiled serum from a mouse model of systemic lupus erythematosus (SLE) and detected both expected and previously unrecognized reactivities. The improved labeling and detection method described here overcomes several problems that have hindered antigen protein microarrays and should facilitate translation to the clinical setting.

Using the two-color Fab labeling method we found that we could improve intraslide and interslide reproducibility and reliably detect changes in autoreactivity. To test the two-color Fab labeling method in a disease setting, we profiled the autoantibody response in a mouse model of SLE. The novel two-color Fab-labeling method addresses difficulties that have confronted autoantigen microarrays and represents an important advance toward applying this platform to the clinical setting.

Results

Two-color Fab labeling for probing autoantigen microarrays. In order to test whether two-color Fab labeling could differentiate serum samples on the same array, we spiked mouse monoclonal anti-myeloperoxidase (anti-MPO) or mouse monoclonal anti-proteinase 3 (anti-PR3) into normal mouse serum. We pre-incubated these spiked samples with cyanine-3 (Cy3) or cyanine-5 (Cy5) labeled goat anti-mouse (GAM) monovalent Fab fragments, respectively. To remove free Fab fragments we passed the mixture over mouse-immunoglobulin G (mlgG)-coated agarose beads in a 0.5 ml spin-column. We then mixed the two samples and applied them to an autoantigen microarray.

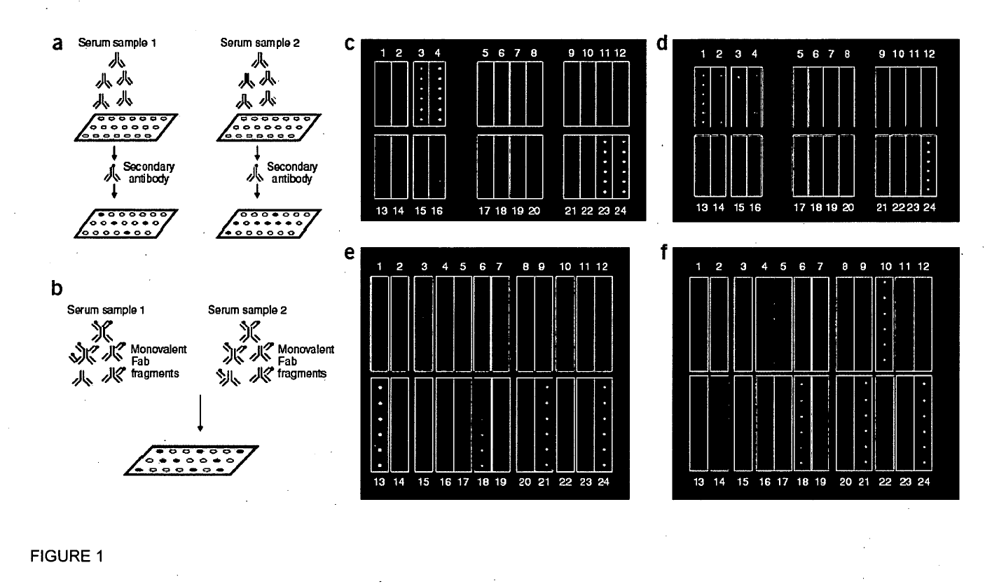

The autoantigen arrays used for these experiments were developed to study a variety of autoimmune disorders, including antineutrophil cytoplasmic antibody (ANCA) positive vasculitides. They were composed of a diverse panel of antigens, including three preparations of myeloperoxidase MPO (MPO-1, MPO-2, and MPO-3) and PR3 (PR3-1, PR3-2, and PR3-3). It has been shown previously that autoantibodies to perinuclear ANCA (pANCA) and cytoplasmic ANCA (cANCA) recognize primarily MPO and PR3 respectively. The scanned images demonstrate that the two-color Fab method qualitatively differentiates the anti-MPO and anti-PR3 reactive sera based on the dominant fluorescence emission at MPO or PR3 features (FIG. 1c).

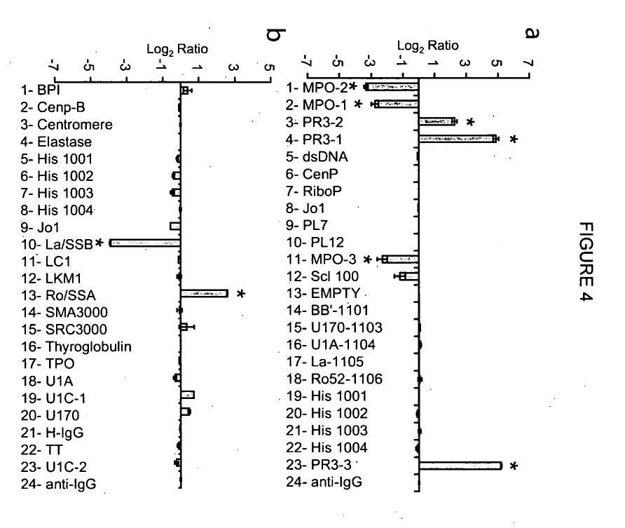

To further validate the technique, we performed a dye-swap in which the samples were each pre-incubated with the alternative fluorophore (FIG. 1d). As the scanned images demonstrate, the reactivities of the two serum samples reflect which fluorophore is used in the labeling reaction. One of the MPO antigens, MPO-3 did not yield as robust a fluorescent signal as the others, perhaps due to purity or concentration. Differences were quantitated by calculating the log₂ of the ratios averaged across both dye-swap experiments and demonstrated changes greater than two-fold for relevant antigens (FIG. 4).

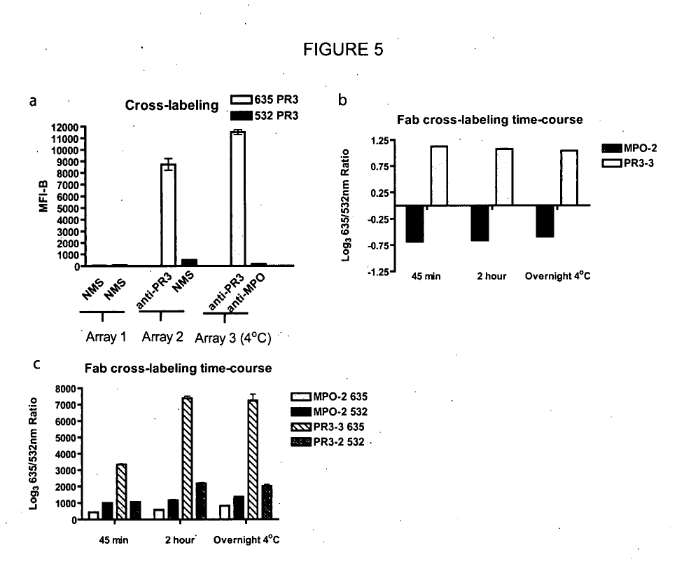

We validated the method for human samples using human anti-Ro and anti-La control sera spiked into normal human serum (FIGS. 1e, f). For these experiments we used goat anti-human (GAH) monovalent Fab fragments conjugated to Alexa Fluor dyes (Alexa647 is a Cy5 equivalent and Alexa555 is a Cy3 equivalent). This experiment showed that the method could be generalized to human studies. One potential drawback of two-color methods is the potential for systematic dye bias, which we did observe in our human studies for antigens such as U1A (FIGS. 1e, f and FIG. 5). Importantly, by averaging data from both dye-swap experiments we were able to identify and greatly reduce this type of artifact during statistical analysis (FIG. 4). Using the Cy3 and Cy5 dyes, which are almost similar structurally, we observed substantially reduced bias for these antigens (Table 3). A mock labeling experiment performed without serum showed no fluorescent signal at any of the array features. These data demonstrate that the two-color Fab-labeling method permits direct comparison of autoantibody profiles on autoantigen microarrays.

TABLE 3

| Antigen | Fluorophore | 635 nm | 532 nm | Ratio | Log2 |
|---|---|---|---|---|---|
| Cathepsin G | Alexa | 15388 | 2554 | 6.0 | 2.6 |
| Histone | Alexa | 600 | 146 | 4.1 | 2.0 |
| U1A | Alexa | 1375 | 200 | 6.9 | 2.8 |
| U1C | Alexa | 2498 | 573 | 4.4 | 2.1 |
| Cathepsin G | Cyanine | 9988 | 3039 | 3.3 | 1.7 |
| Histone | Cyanine | 234 | 144 | 1.6 | 0.7 |
| U1A | Cyanine | 341 | 254 | 1.3 | 0.4 |
| U1C | Cyanine | 801 | 287 | 2.8 | 1.5 |

Bias of Alexa and Cyanine dyes. Chart displays MFI-B from 635 nm and 532 nm channels, median 635/532 nm ratio, and log₂ of the 635/532 nm ratio from a self-self comparison of human serum.

One potential concern with this approach is the possibility of cross-labeling. If Fab fragments dissociate from one sample and associate with the other sample, then this method would not reliably reflect differences in the serum samples. We determined that cross-labeling occurred at a rate of less than 5% at room-temperature or 1% at 4° C., since normal mouse serum (NMS) exhibited minimal anti-PR3 reactivity when probed on an array with near saturating amounts of anti-PR3 antibody (FIG. 5). We observed similar results using human serum with anti-Ro and anti-La reactivity. Moreover, a time-course experiment demonstrated stable ratios and fluorescent intensities when different samples are incubated on arrays for up to two hours at room temperature or overnight at 4° C. (FIG. 5).

Reproducibility of single-color and two-color approaches. We hypothesized that two-color data would be subject to less interslide and intraslide variability than single-color data since it helps control for spot-to-spot and array-to-array variability. We spiked mouse monoclonal anti-PR3 antibody into normal mouse serum and aliquoted it into two separate pools for “self-self” comparisons. Although the median of ratios and the median fluorescent intensity (MFI) minus background (B) are entirely different measurements, the coefficient of variance (% CV) allows the two to be compared with respect to variability (Table 1). In aggregate, the interslide and intraslide % CVs for the two-color Fab method are significantly lower than the % CVs for the conventional single-color method when using as few as three replicate features or as many as twelve replicate features (Table 1). Despite equivalently high variability in the MFI-B for the two-color method, the median of ratios exhibited low % CV (Table 1). Additionally, the two-color Fab method allows for reliable detection of three-fold changes in relative autoantibody levels (Table 4), indicating that even relatively subtle differences can be reproducibly measured by the two-color Fab method.

TABLE 1

| | | Interslide | | | | Intraslide | | | |
|---|---|---|---|---|---|---|---|---|---|
| | | Single-Color | | Two-Color | | Single-Color | | Two-Color | |
| Replicates | Antigen | MFI-B | % CV | MR | % CV* | MFI-B | % CV | MR | % CV** |
| 12 | PR3-1 | 2477 | 19% | 0.88 | 8% | 2399 | 15% | 0.94 | 4% |
| | PR3-2 | 3263 | 18% | 0.89 | 7% | 2805 | 19% | 0.94 | 5% |
| | PR3-3 | 1840 | 13% | 0.91 | 4% | 1643 | 13% | 0.94 | 3% |
| 6 | PR3-1 | 2432 | 20% | 0.88 | 8% | 2442 | 12% | 0.94 | 4% |
| | PR3-2 | 3278 | 18% | 0.89 | 7% | 2851 | 16% | 0.95 | 7% |
| | PR3-3 | 1832 | 13% | 0.91 | 4% | 1628 | 17% | 0.95 | 3% |
| 3 | PR3-1 | 2214 | 30% | 0.88 | 7% | 2178 | 20% | 0.92 | 6% |
| | PR3-2 | 2946 | 17% | 0.88 | 7% | 2483 | 11% | 0.92 | 6% |
| | PR3-3 | 1825 | 12% | 0.90 | 6% | 1661 | 21% | 0.95 | 2% |

*p < 0.000.1 Single-Color versus Two-Color Interslide % CV. Paired t-test.
**p < 0.0001 Single-Color versus Two-Color Intraslide % CV. Paired t-test.

Intraslide and interslide variability. We spiked mouse monoclonal anti-PR3 antibody (0.2 ug) into normal mouse serum (2 ul containing 20 ug total IgG) for “self-self” comparisons by the single-color or two-color Fab method. The MFI with background subtracted (MFI-B) at 532 nm emission is reported for single-color data, and the Median of Ratios (MR) is reported for two-color data, both normalized to IgG. We analyzed twelve, six, and three replicates of each antigen on the arrays.

TABLE 4
Measurement of artificial up- and down-regulation of antibody levels. We spiked
mouse monoclonal anti-PR3 antibody into normal mouse serum in three amounts:
600 ng (3×), 200 ng (1×) or 66 ng (0.33×) in normal mouse serum (2
ul containing 20 ug total IgG). All three samples (3×, 1×, 0.33×)
were then compared to a reference mixture with a 1× concentration of anti-PR3
antibody using both single-color and two-color methods. Ratios and corresponding
95% confidence intervals (n = 12) are displayed.

| Method | Antigen | 3× | 1× | 0.33× |
|---|---|---|---|---|
| Single-color | PR3-1 | 1.34 (0.83 to 2.14) | 1.62 (1.28 to 2.06) | 0.37 (0.25 to 0.56) |
| | PR3-2 | 2.91 (2.69 to 3.15) | 1.01 (0.95 to 1.07) | 0.48 (0.45 to 0.52) |
| | PR3-3 | 1.93 (1.52 to 2.45) | 2.14 (1.74 to 2.63) | 0.33 (0.27 to 0.40) |
| Two-color | PR3-1 | 2.22 (2.14 to 2.29) | 1.00 (0.98 to 1.02) | 0.29 (0.28 to 0.30) |
| | PR3-2 | 1.99 (1.93 to 2.06) | 1.00 (0.98 to 1.02) | 0.45 (0.44 to 0.47) |
| | PR3-3 | 2.31 (2.25 to 2.38) | 0.96 (0.93 to 0.99) | 0.29 (0.28 to 0.31) |

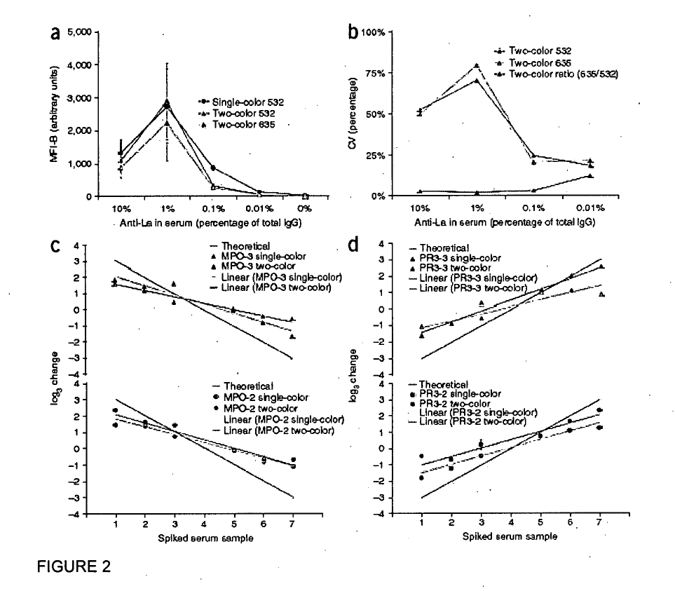

Signal intensity, sensitivity, and dynamic range. Autoantigen arrays have previously proven to be similar to conventional ELISA with respect to sensitivity, specificity, and dynamic range. The fluorescent signal from antibodies labeled with fluorescently-tagged Fab fragments, however seems generally to be weaker in intensity than detection with secondary reagents. To determine the signal intensity and dynamic range of the two labeling approaches we spiked monoclonal anti-La into normal mouse serum in serial ten-fold dilutions (FIG. 2a). At the highest concentration of anti-La (2 μg of anti-La in 2 μl of serum containing 20 μg of total IgG), the fluorescence signal was paradoxically low for both methods, as previously described for saturated antibody assays. Both methods detected anti-La reactivity at 0.1% and 0.01% of the serum IgG, with overall MFI and over half of the pixels at least two standard deviations above background (FIG. 2a). The dynamic range of both methods was comparable, and was linear over approximately two orders of magnitude. One interesting finding is that the MFI-B seemed to have the largest error when the signal was also the largest (1% anti-La in serum), while the error at more dilute concentrations was smaller (0.1% anti-La in serum) (FIG. 2b). Although the % CV of the MFI-B appeared to depend dramatically on concentration and/or signal intensity, the % CV of the ratio was similar at all anti-La concentrations tested (FIG. 2b). Additionally, the % CV of the ratio was lower than the % CV of the MFI-B at all anti-La concentrations tested. When the goal is to detect subtle changes over a wide range of concentrations a two-color method is preferable.

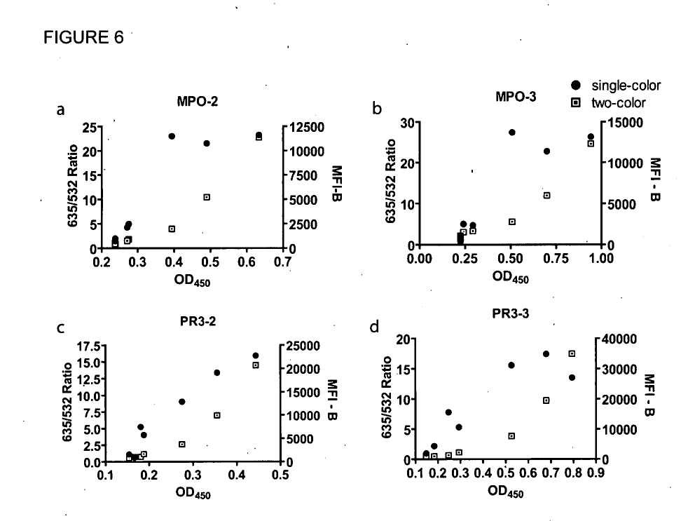

To compare the sensitivity, specificity, and dynamic range of single-color and two-color Fab methods for measuring changes in autoantibody levels, we spiked anti-MPO and anti-PR3 into serum at serial three-fold dilutions from approximately 10% of serum IgG down to approximately 0.01% of serum IgG, representing a 3⁶- or 729-fold change in concentration. We designed the seven serum samples with the gradient of anti-MPO and anti-PR3 reactivities in opposing directions, so that the sample with the highest anti-MPO reactivity had the lowest anti-PR3 reactivity and vice versa. We calculated the log₃ change relative to the middle value (anti-MPO and anti-PR3 at approximately 0.3% of total serum IgG) and fit the data by linear regression for each antigen (FIGS. 2c, d). While there was no statistically significant difference in the slopes between two-color and single-color methods, the two-color method had a significantly higher R² value than the single-color method (Table 2). We also compared both the single-color and two-color data to conventional ELISA performed on the same samples and determined that the two-color method had better correlation with ELISA than the single-color method (Table 2 and FIG. 6). Although both methods underestimated changes in autoreactivity, the two-color method provided data that was significantly more linear and better correlated with ELISA than the single-color method.

TABLE 2

| | Single-color | Two-color |
|---|---|---|
| slope | 0.56 ± 0.04 | 0.51 ± 0.04* |
| R² | 0.84 ± 0.02 | 0.95 ± 0.01** |
| Spearman r with ELISA | 0.88 ± 0.02 | 0.97 ± 0.02*** |

*n.s. paired t test
**p = 0.01 paired t test
***p = 0.02 Wilcoxon matched pairs test

Artificial antibody up- and down-regulation measured by single-color and two-color Fab methods. Slope, regression coefficient, and nonparametric correlation (Spearman r) with conventional ELISA for single-color and two-color Fab methods. Error reported as s.e.m., n=3.

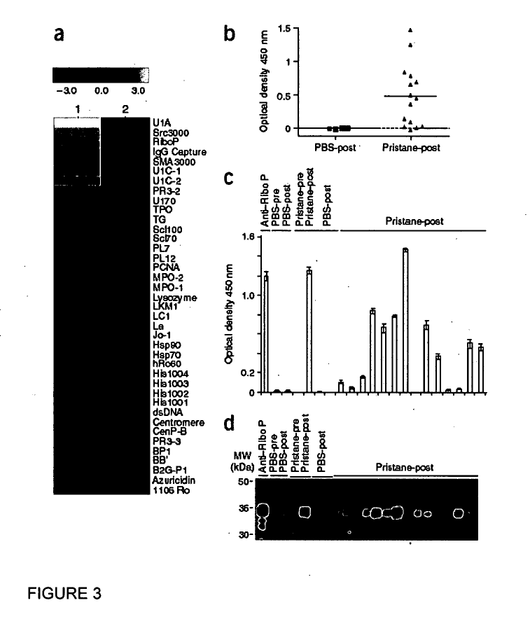

Two-color Fab method identifies Ribo P autoantibodies in SLE model. In order to validate the two-color Fab labeling method in a disease model, we analyzed serum samples from the pristane model of lupus. The arrays contained 468 features with a redundancy of 12 replicates per antigen, including both common and uncommon autoantigens for a variety of autoimmune diseases, as well as several features used for standardization and quality control. Serum from a pristane-treated BALB/c mouse 20 weeks after induction (Pristane-post) was labeled with Alexa647-Fab fragments and compared to Alexa555-Fab labeled serum from the same mouse obtained immediately prior to induction (Pristane-pre) (FIG. 3a). As a negative control serum from a PBS-treated BALB/c mouse 20 weeks after mock induction (PBS-post) (Alexa647-Fab) was compared to serum from the same mouse obtained immediately prior to mock induction (PBS-pre) (Alexa555-Fab) (FIG. 3a). We repeatedly observed reactivity to autoantigens known to be targeted in the pristane model, such as U1A, U1C, and dsDNA, (FIG. 3a). We also reproducibly detected strong reactivity to Ribo P, which was not anticipated (FIG. 3a).

Most of the autoantibodies that we detected using the two-color autoantigen arrays have been previously reported in pristane-treated BALB/c mice, but reactivity to Ribo P had not previously been detected. Autoantibodies to the ribosomal P phosphoproteins are characteristic of SLE and are typically directed against three proteins, P0, P1 and P2 (35 kD, 19 kD, and 17 kD respectively). Previous studies suggested that these autoantibodies target a conserved 22-amino acid sequence at the carboxyl-terminus that is shared by all three proteins, but the reactivity may involve other epitopes.

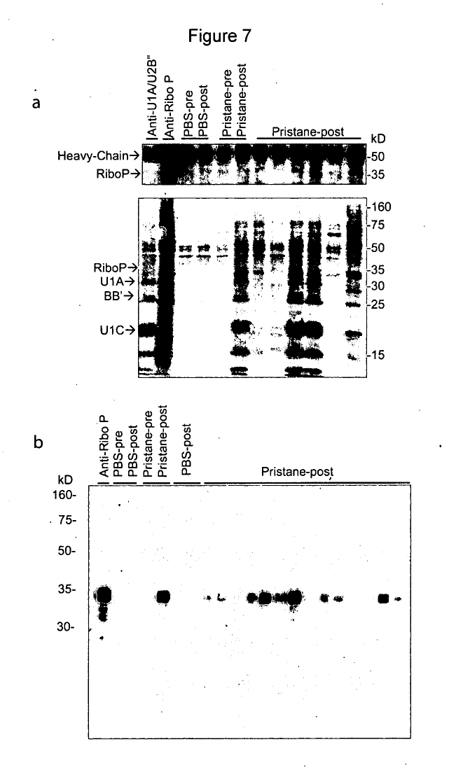

Serum from one pristane-treated BALB/c mouse demonstrated strong, reproducible reactivity to a recombinant Ribo P0 that was used on the arrays (FIG. 3a). Subsequent single-color array analysis also identified reactivity to Ribo P in pristane treated BALB/c mice. Although these data were from a single mouse, this unexpected reactivity encouraged us to investigate a larger panel of pristane-treated BALB/c mice. By conventional ELISA 9/15 (60%) exhibited strong reactivity, while 5/15 (33%) exhibited lower reactivity to recombinant P0 (FIG. 3b). Mice from the PBS-treated group lacked such autoantibodies (FIG. 3b). To rule-out contamination as a cause of this reactivity, the P0 antigen used for the ELISA and the arrays was fractionated by sodium dodecyl sulfate polyacrylamide gel electrophoresis (SDS-PAGE), transferred to a nitrocellulose membrane, and probed using serum from each pristane-treated or PBS-treated mouse (FIG. 3c). A band at 35 kD corresponding to P0 was detectable in all of the mice that tested positive by ELISA, arguing that this reactivity was indeed specific for P0 (FIG. 3c). BALB/c mice primed with pristane had previously appeared negative for Ribo P reactivity by immunoprecipitation of radiolabeled extract and ELISA using the C-terminal 22 amino acid peptide. Consistent with these previous studies, we did not observe reactivity to Ribo P when we immunoprecipitated radiolabeled EL4 cell extract with serum from pristane-treated BALB/c mice (FIG. 7a). The anti-Ribo P0 response in BALB/c mice primed with pristane, although positive by ELISA and western blot, does not immunoprecipitate the protein.

While there has been extensive effort in the field of transcript profiling to examine sources of error and variability, these issues have yet to be addressed in a systematic manner for protein arrays, particularly autoantigen microarrays. While it is true that some popular transcript profiling platforms employ single-color labeling methods, these platforms often have rigorous quality control in fabrication and design that minimize variability. Antigen arrays, however, are being developed for vastly different macromolecular species (lipids, proteins, carbohydrates, nucleic acids), widely variable molecular sizes (peptides, protein complexes), variable sample complexity (recombinant or affinity-purified proteins), and variable sample storage buffer (glycerol, PBS, other buffers), which complicate array production. Two-color methods control for many sources of variability by allowing two samples to bind the same feature on the same array. We found that our rapid two-color labeling method using Fab fragments improved reproducibility and linearity over a wide range of antibody level changes. Using the two-color Fab labeling method we profiled autoantibody levels and discovered a previously unreported reactivity to Ribosomal P0 in the pristane model of SLE in BALB/c mice. This finding validated the technology for profiling humoral immune response changes during disease onset. We believe that ultimately the two-color Fab labeling approach will facilitate the study of more subtle changes in autoantibody profiles, such as monitoring the response to therapy over time in an individual patient.

One potential drawback to the two-color Fab-labeling method is that the fluorophore-Fab fragments are not covalently attached to the sample, allowing for the possibility of mobility during the experiment. However, this was not a problem with the methods of the invention (FIG. 5). A second potential problem is systematic dye bias, which is a universal concern of two-color labeling approaches. This bias can be minimized by averaging dye-swap experiments, using cyanine instead of Alexa dyes, or using a constant reference. Taken as a whole, the improvement in reproducibility of this novel two-color Fab-labeling method addresses problems facing autoantigen microarray technology and will help transition autoantibody profiling into a reliable clinic tool.

Methods

Probing and scanning of autoantigen arrays. We blocked the autoantigen arrays with 3% fetal calf serum (FCS) and 0.05% Tween 20 (Sigma Chemical Co., St. Louis, Mo.) in phosphate buffered saline (PBS) (GIBCO, Grand Island, N.Y.) either for one hour at room temperature or overnight at 4° C.

We then probed these blocked slides by either the single-color or two-color Fab methods. Single-color arrays were probed as previously described. Briefly, we incubated the arrays for one hour at 4° C. with 2 ul of serum diluted in 1 ml of 3% FCS and 0.05% Tween 20 in PBS (PBST). We then washed the slides twice for twenty minutes in 3% FCS PBST. We incubated the slides with either a Cy3 conjugated donkey anti-human or goat anti-mouse secondary antibody (Jackson ImmunoResearch, West Grove, Pa.) at a dilution of 1:1,000 for one hour at 4° C. After incubation, we washed the slides twice for thirty minutes in 3% FCS PBST then twice for twenty minutes in PBS, rinsed them for ten seconds in double-distilled deionized water (ddH₂O), centrifuged them to dryness at room-temperature for five minutes, and scanned them.

For the two-color Fab labeling method, we first pre-incubated the serum and Fab fragments for ten to thirty minutes at room-temperature. Unless otherwise stated, we labeled the serum at an Fab:IgG molar ratio of 4.5:1 during pre-incubation. We added 150-350 μl of whole IgG coupled agarose beads (Jackson ImmunoResearch) to empty 0.5 ml Zeba spin columns (Pierce, Rockford, Ill.). We added the serum-Fab mixture to the column and incubated at room temperature for five to ten minutes before centrifugation for one minute at 10,000×g. Alternatively, we pre-spun the beads in spin-columns to remove the aqueous phase, and then added the serum-Fab mixture to the packed beads. We placed the flow-through from two labeling reactions on ice and diluted to a final volume of 1 ml of 3% FCS in PBST. We then applied this mixture to the slides for an incubation period of 45 minutes at 4° C. unless otherwise indicated. After incubation, we washed the slides three times for five minutes in 3% FCS PBST, then five minutes in PBS, rinsed them for ten seconds in double-distilled deionized water (ddH₂O), centrifuged them to dryness at room-temperature for five minutes, and then scanned them.

Data analysis. We used the GenePix 4000 scanner to scan the arrays and the GenePix Pro Version 5.0 software (Molecular Devices, Union City, Calif.) to analyze the images. For analysis, we used either the Median Fluorescent Intensity (MFI) minus background (B) or the Median of 635 nm/532 nm ratios as indicated. We applied a low-intensity cut-off filter during data analysis to exclude any spots where the intensity in over half of the pixels is less than two standard deviations above background for both 635 nm and 532 nm channels. We normalized the ratios at each feature to the ratio of total IgG between the two samples. To determine the ratio of IgG levels we used the Easy-Titer IgG Assay Kit (Pierce, Rockford, Ill.) or assumed a ratio of 1.0 in self-self experiments. We multiplied all ratios by the correction factor (IgGᵣₐₜᵢₒ_{_{—}}ₜₒₜₐₗ/IgG_{635 nm/532 nm}) where IgGᵣₐₜᵢₒ_{_{—}}ₜₒₜₐₗ is the ratio of total IgG for the two samples determined prior to probing the microarray and IgG_{635 nm/532 nm} is the ratio observed at the anti-IgG capture antibody feature. For single-color data, MFI-B for each feature was normalized to the MFI-B for anti-IgG. After filtering out low-intensity data and normalizing to the ratio of total IgG, the mean of the ratios or the log₂ of the ratios was calculated.

What is claimed is:

1. A method for labeling antigen, the method comprising:
labeling a first Fab reagent specific for antibodies present in a patient sample with a detectable label;
labeling a second Fab reagent specific for antibodies present in a patient sample with a second detectable label, wherein said first detectable label and said second detectable label are spectrally resolved;
contacting a first patient sample containing antibodies with said first Fab reagent;
contacting a second patient sample containing antibodies with said second Fab reagent;
contacting antigen with said first and said second patient samples to label said antigen.

2. The method according to claim 1, wherein at least one of said first and said second detectable labels are fluorochromes.

3. The method according to claim 2, wherein both said first and said second detectable labels are fluorochromes.

4. The method according to claim 1, wherein said patient samples contains autoantibodies.

5. The method according to claim 1, wherein said first and said second patient samples are the same.

6. The method according to claim 1, wherein said first and said second patient samples are different.

7. The method according to claim 6, wherein said first and said second patient samples are a test sample and a reference sample.

8. The method according to claim 6, wherein said first and said second patient samples are obtained from a patient at different periods of time.

9. The method according to claim 1, wherein said patient is a human.

10. The method according to claim 1, wherein said antigen is present in an array.

11. The method according to claim 10, wherein said array comprises tissue sections.

12. The method according to claim 10, wherein said array comprises purified antigens.

13. The method according to claim 12, wherein said antigens include autoantigens.

14. The method according to claim 10, wherein said array comprises cells.

15. The method according to claim 1, further comprising the steps of labeling at least one Fab reagent with additional detectable reagent(s);
contacting one or more patient samples with said Fab reagent;
contacting antigen with said patient samples.

16. The method according to claim 1, wherein at least one of said Fab reagents binds to substantially all of the antibodies present in said patient sample.

17. The method according to claim 1, wherein at least one of said Fab reagents binds to a subset of antibodies present in said patient sample.
